(19) United States
(12) Patent Application Publication
(10) Pub. No.: US 2022/0404044 A1
(43) Pub. Date: Dec. 22, 2022

(54) ENERGY-TRANSMISSIBLE AIR FILTER SYSTEM AND METHOD

(71) Applicant: David Louis Kirschman, Dayton, OH (US)

(72) Inventor: David Louis Kirschman, Dayton, OH (US)

(73) Assignee: Aerobiotix, Inc., Miamisburg, OH (US)

(21) Appl. No.: 17/354,299

(22) Filed: Jun. 22, 2021

(51) Int. Cl. F24F 8/22; B01D 46/52; B01D 46/00; A61L 2/10; A61L 9/20; F24F 8/108

(52) U.S. Cl. CPC F24F 8/22; B01D 46/52; B01D 46/0002; B01D 46/0028; A61L 2/10; A61L 9/20; F24F 8/108; B01D 2321/343; A61L 2209/16

(57) ABSTRACT

An energy-transmissible air filtration system having at least one mechanical filter and at least one electromagnetic energy source. The electromagnetic energy source generates an electromagnetic energy wave or force that traverses the at least one mechanical filter. This results in substantially simultaneous mechanical filtration and energy-based decontamination of an airstream. Other features of the embodiments are also shown.

### BACKGROUND OF THE INVENTION

### Field of the Invention

This invention relates to decontamination systems and, more particularly, to an energy transmission air filter and system that permits substantially simultaneous filtering and energy decontamination of an airstream.

### 2. DESCRIPTION OF THE RELATED ART

The need for air filtration is ubiquitous in the modern world. A myriad of technologies have been developed to remove or inactivate biological and non-biological contaminants from an airstream. Current removal technologies fall into two general categories: mechanical filtration and energy-based decontamination. Mechanical filtration can include sieve filtration with a filter membrane, a solid filter media chamber, or high efficiency particulate arrestance (HEPA) filtration which uses aerodynamic forces to trap contamination on a media surface. Filtration media can consist of any physical form which can interact with air contaminants, such as paper, polymer, metal, or mineral. Mechanical filtration is particularly important in the removal of inorganic contaminants such as dust and particulates. A challenge of mechanical filtration is that the filter material itself can become contaminated, reducing system efficiency.

Energy-based decontamination methods include application of an electromagnetic particle or wave to an airstream. A challenge in the application of energy-based decontamination, is that adequate irradiation requires a certain dwell time in order to achieve an inactivation dosage. The dosage given is linearly related to dwell time. However, in a fast-moving airstream, it is difficult to achieve adequate dwell times.

A critical requirement of an air decontamination system is the ability to eliminate airborne bioaerosols. In order to maximize energy dosage to the airstream, it is advantageous to stall the aerosol within the energy field for as long as possible, without reducing the overall airflow rate, and hence system efficiency.

In the prior art, there are several examples or irradiation of external filter surfaces, however these systems do not address dwell time lengthening, or the internal penetration of the filter body.

What is needed, therefore, is a filter system and material that improve over the shortcomings of the prior art.

### SUMMARY OF THE INVENTION

In order to address these requirements and challenges, one object of the invention is to provide a new form of mechanical filter having a filter material that is substantially transmissible to the application of energy based decontamination or irradiation.

What is further needed is a transmissible media which serves to attract and immobilize bioaerosol droplets through surface interactions between the bioaerosol droplet and a hydrophilic surface.

One object of the invention is to provide a filter and filter material that permits substantially simultaneous irradiation by an energy-based decontamination electromagnetic particle or wave to the airstream as it flows through the filter. This application may be ultraviolet irradiation, however, other phenomena including, infrared, electrons, electrostatics, charged particles, ions, ionizing radiation, microwaves, visible light and other electromagnetic forces can be applied. Energy-based decontamination is important in the removal or inactivation of biological condiments, such as viruses, bacteria, fungi, cells, and spores.

Another object of the invention is to provide to provide at least one mechanical filter that comprises a filter material that is substantially transmissible to the application of energy-based decontamination or irradiation.

Still another object of the invention is to provide a filter material comprising a transmissible media that serves to attract and immobilize bioaerosol droplets.

Yet another object of the invention is to provide a transmissible media that attracts and immobilizes bioaerosol droplets through surface interactions between the bioaerosol droplets and a hydrophilic surface of the filter material.

Still another object of the invention is to provide an energy-transmissible air filtration system having at least one transmissible filter and at least one energy source, wherein the at least one transmissible filter is adapted to permit electromagnetic energy waves or forces to traverse the at least one transmissible filter, thereby resulting in a substantially simultaneous or simultaneous mechanical filtration and energy-based decontamination of an airstream.

Yet another object of the invention is to provide a mechanical filter having at least one transmissible filter surface that functions to aerodynamically influence or entrap air contaminants such that the contaminants are transiently or permanently captured within the at least one transmissible filter.

Another object of the invention is to provide a filter having a volume that is substantially transmissible to electromagnetic energy such that the radiation from the electromagnetic energy substantially permeates the volume.

Yet another object of the invention is to provide a system that utilizes electromagnetic radiation comprising the ultraviolet or near ultraviolet spectrum between 10-420 nm wavelength.

Still another object of the invention is to provide a filter comprising a substantially hydrophilic surface with a water contact angle of less than about 40 degrees and/or a critical surface tension greater than 40 dynes/cm.

Yet another object of the invention is to provide a filter having a material having a substantially hydrophilic surface adapted to increase a surface to bioaerosol interaction, exposure, and/or dwell time within the electromagnetic field generated by an electromagnetic energy radiation generator.

In one aspect, one embodiment of the invention comprises an energy-transmissible air filtration system comprising at least one transmissible filter comprising a filter material that is adapted to permit a substantially transmissible application of decontamination energy adapted to decontaminate an airstream having contaminants flowing though the at least one transmissible filter; wherein the decontamination energy traverses an interior area of the filter in order to apply the decontamination energy substantially simultaneously as the filter material mechanically filters the airstream as the decontamination energy is applied.

In another aspect, another embodiment of the invention comprises an energy-transmissible air filtration system comprising a housing having an inlet and an outlet adapted to permit an airstream to pass therethrough; an decontamination energy generator for generating electromagnetic energy effective to remove at least a portion of biological contaminants in the airstream; and at least one transmissible filter having a transmissible filter material adapted to permit the electromagnetic waves to substantially pass therethrough, the at least one transmissible filter being defining an internal area having a predetermined volume; the decontamination energy generator being situated in operative relationship with the at least one transmissible filter is substantially transmissible such that the electromagnetic energy may pass therethrough while the decontamination energy generator substantially simultaneously the decontamination energy generator applies the electromagnetic energy to the airstream as it passes through the filter.

In still another aspect, another embodiment of the invention comprises an energy-transmissible filter comprising a filter housing; and a filter material located in the housing; the filter material permitting electromagnetic energy to traverse therethrough to facilitate decontaminating an airstream passing through the filter as the filer material mechanically filters the filter material.

In yet another aspect, another embodiment of the invention comprises an energy-transmissible air filtration system comprising at least one mechanical filter; and at least one energy source operatively associated with the at least one mechanical filter for generating an electromagnetic energy wave or force; wherein the electromagnetic energy wave or force traverses inside at least one material the filter, resulting in simultaneous mechanical filtration and energy-based decontamination of an airstream as the airstream passes through the filter.

This invention, including all embodiments shown and described herein, could be used alone or together and/or in combination with one or more of the features covered by one or more of the following list of features:

The energy-transmissible air filtration system wherein the filter material is adapted to at least one of aerodynamically influence the contaminants in the airstream of entrap contaminants such that the contaminants are transiently or permanently captured within the filter material.

The energy-transmissible air filtration system wherein at least one transmissible filter comprises a filter body having an exterior surface, the filter body defining the interior area to have a predetermined volume; the filter material permitting the decontamination energy to be substantially transmissible in order to permeate the interior area and the predetermined volume.

The energy-transmissible air filtration system wherein the filter material comprises at least one of silicate, quartz, glass, textiles, cellulosic materials, fluorinated polymers, silicon nitride, glass, silver, copper, aluminum, iron, titanium, titanium dioxide, ferric oxide, tin oxide, further comprising solid, woven, planar, folded, or discrete elements.

The energy-transmissible air filtration system wherein the decontamination energy comprises at least one of: a non-ionizing radiation, an ionizing radiation, an ultraviolet, an infrared, electrons, electrostatics, a plasma, a light, a laser, an LED, a lamp, an excited gas, and similar phenomena which function to effect biological and non-biological contamination within the airstream.

The energy-transmissible air filtration system wherein the decontamination energy is generated by a decontamination energy generator that generates the decontamination energy such that it substantially fills or traverses the predetermined volume.

The energy-transmissible air filtration system wherein the decontamination energy is generated by a decontamination energy generator that generates the decontamination energy such that it substantially fills or traverses the predetermined volume.

The energy-transmissible air filtration system wherein the decontamination energy is applied by a decontamination energy generator, the decontamination energy generator generates ultraviolet or near ultraviolet spectrum between 10-420 nm wavelength.

The energy-transmissible air filtration system wherein at least one of the filter material or the internal area comprises a material that permits the spectrum to be substantially transmissible therethrough.

The energy-transmissible air filtration system wherein filter material comprises a hydrophilic surface with a water contact angle of less than about 40 degrees.

The energy-transmissible air filtration system wherein filter material comprises a hydrophilic surface having a surface tension greater than 40 dynes/cm in order to facilitate increasing at least one of a surface-bioaerosol interaction or an exposure or dwell time within the electromagnetic field.

The energy-transmissible air filtration system wherein filter material comprises a hydrophilic surface having a surface tension greater than 40 dynes/cm in order to facilitate increasing at least one of a surface-bioaerosol interaction or an exposure or dwell time within the electromagnetic field.

The energy-transmissible air filtration system application of electromagnetic energy sufficient to substantially decontaminate the airstream having contaminants flowing though the filter as the filter material mechanically filters the air stream.

The energy-transmissible air filtration system wherein the filter material is adapted to at least one of aerodynamically influence the contaminants in the airstream of entrap contaminants such that the contaminants are transiently or permanently captured within the filter material.

The energy-transmissible air filtration system wherein at least one transmissible filter comprises a filter body having an exterior surface, the filter body defining the interior area to have a predetermined volume; the filter material permitting the decontamination energy to be substantially transmissible in order to permeate the interior area and the predetermined volume.

The energy-transmissible air filtration system wherein the filter material comprises at least one of: silicate, quartz, glass, textiles, cellulosic materials, fluorinated polymers, silicon nitride, glass, silver, copper, aluminum, iron, titanium, titanium dioxide, ferric oxide, tin oxide, further comprising solid, woven, planar, folded, or discrete elements.

The energy-transmissible air filtration system wherein the decontamination energy comprises at least one of: a non-ionizing radiation, an ionizing radiation, an ultraviolet, an infrared, electrons, electrostatics, a plasma, a light, a laser, an LED, a lamp, an excited gas, and similar phenomena which function to effect biological and non-biological contamination within the airstream.

The energy-transmissible air filtration system wherein the decontamination energy is generated by a decontamination energy generator that generates the decontamination energy such that it substantially fills or traverses the predetermined volume.

The energy-transmissible air filtration system wherein the decontamination energy is generated by a decontamination energy generator that generates the decontamination energy such that it substantially fills or traverses the predetermined volume.

The energy-transmissible air filtration system wherein the decontamination energy is applied by a decontamination energy generator, the decontamination energy generator generates ultraviolet or near ultraviolet spectrum between 10-420 nm wavelength.

The energy-transmissible air filtration system wherein at least one of the filter material or the internal area comprises a material that permits the spectrum to be substantially transmissible therethrough.

The energy-transmissible air filtration system wherein filter material comprises a hydrophilic surface with a water contact angle of less than about 40 degrees.

The energy-transmissible air filtration system wherein filter material comprises a hydrophilic surface having a surface tension greater than 40 dynes/cm in order to facilitate increasing at least one of a surface-bioaerosol interaction or an exposure or dwell time within the electromagnetic field.

The energy-transmissible air filtration system wherein filter material comprises a hydrophilic surface having a surface tension greater than 40 dynes/cm in order to facilitate increasing at least one of a surface-bioaerosol interaction or an exposure or dwell time within the electromagnetic field.

The energy-transmissible filter wherein the filter material is adapted to at least one of aerodynamically influence the contaminants in the airstream of entrap contaminants such that the contaminants are transiently or permanently captured within the filter material.

The energy-transmissible filter wherein at least one transmissible filter comprises a filter body having an exterior surface, the filter body defining the interior area to have a predetermined volume; the filter material permitting the decontamination energy to be substantially transmissible in order to permeate the interior area and the predetermined volume.

The energy-transmissible filter wherein the filter material comprises at least one of silicate, quartz, glass, textiles, cellulosic materials, fluorinated polymers, silicon nitride, glass, silver, copper, aluminum, iron, titanium, titanium dioxide, ferric oxide, tin oxide, further comprising solid, woven, planar, folded, or discrete elements.

The energy-transmissible filter wherein the decontamination energy comprises at least one of: non-ionizing radiation, ionizing radiation, ultraviolet, infrared, electrons, electrostatics, plasma, light, laser, LED, lamp, excited gas, and similar phenomena which function to effect biological and non-biological contamination within the airstream.

The energy-transmissible filter wherein the decontamination energy is generated by a decontamination energy generator that generates the decontamination energy such that it substantially fills or traverses the predetermined volume.

The energy-transmissible filter wherein the decontamination energy is generated by a decontamination energy generator that generates the decontamination energy such that it substantially fills or traverses the predetermined volume.

The energy-transmissible filter wherein the decontamination energy is applied by a decontamination energy generator, the decontamination energy generator generates ultraviolet or near ultraviolet spectrum between 10-420 nm wavelength.

The energy-transmissible filter wherein at least one of the filter material or the internal area comprises a material that permits the spectrum to be substantially transmissible therethrough.

The energy-transmissible filter wherein filter material comprises a hydrophilic surface with a water contact angle of less than about 40 degrees.

The energy-transmissible filter wherein filter material comprises a hydrophilic surface having a surface tension greater than 40 dynes/cm in order to facilitate increasing at least one of a surface-bioaerosol interaction or an exposure or dwell time within the electromagnetic field.

The energy-transmissible filter wherein filter material comprises a hydrophilic surface having a surface tension greater than 40 dynes/cm in order to facilitate increasing at least one of a surface-bioaerosol interaction or an exposure or dwell time within the electromagnetic field.

The energy-transmissible filter wherein the filter material comprises a surface adapted to have a predetermined water contact angle of about 40 degrees or less.

The energy-transmissible filter wherein the filter material comprises a surface tension of at last 40 dynes/cm.

The energy-transmissible air filtration system wherein the at least one mechanical filter comprising at least one transmissible filter surface functioning to aerodynamically influence or entrap air contaminants such that the contaminants are transiently or permanently captured within the filter; the mechanical filter further comprising a filter exterior and a filter body comprising a filter volume, the volume being substantially transmissible to the electromagnetic energy such that the radiation substantially permeates the volume.

The energy-transmissible air filtration system wherein the filter comprising materials including silicate, quartz, glass, textiles, cellulosic materials, fluorinated polymers, silicon nitride, glass, silver, copper, aluminum, iron, titanium, titanium dioxide, ferric oxide, tin oxide, further comprising solid, woven, planar, folded, or discrete elements.

The energy-transmissible air filtration system wherein the electromagnetic energy comprising non-ionizing radiation, ionizing radiation, ultraviolet, infrared, electrons, electrostatics, plasma, light, laser, LED, lamp, excited gas, and similar phenomena which function to effect biological and non-biological contamination within an airstream.

The energy-transmissible air filtration system wherein the electromagnetic energy is detectable throughout substantially all of the filter volume, creating an electromagnetic field substantially filling or traversing the filter volume.

The energy-transmissible air filtration system wherein the electromagnetic radiation comprising the ultraviolet or near ultraviolet spectrum between 10-420 nm wavelength.

The energy-transmissible air filtration system wherein the at least one transmissible filter surface comprising a substantially hydrophilic surface with a water contact angle less than 40 degrees and/or a critical surface tension greater than 40 dynes/cm. The hydrophilic surface increasing surface-bioaerosol interaction, exposure, and/or dwell time within the electromagnetic field.

The energy-transmissible air filtration system wherein the filter volume comprising a material substantially transmissible to the spectrum.

This invention, including all embodiments shown and described herein, could be used alone or together and/or in combination with one or more of the features covered by one or more of the following list of features:

These and other objects and advantages of the invention will be apparent from the following description, the accompanying drawings and the appended claims.

### BRIEF DESCRIPTION OF THE ACCOMPANYING DRAWINGS

FIG. 1 is view of a prior art decontamination system showing a non-transmissible filter that filters an airstream after radiation has been applied to the airstream;

FIG. 2 is a view of one embodiment of the invention showing an electromagnetic radiator radiating a filter and filter material simultaneously as the filter mechanically filters the airstream;

FIG. 2A is a view showing various features of the embodiment shown in FIG. 2;

FIG. 2B shows the embodiment of FIG. 2A but without a pre-chamber or post-chamber;

FIG. 2C is an embodiment similar to FIG. 2A showing a perforated window as opposed to a transparent window of the type shown in FIG. 2A;

FIG. 3 is a view of a prior art a bioaerosol contaminant droplet illustrating a low surface tension of a filter material;

FIG. 4 is a view of a current embodiment showing the filter material having a high surface tension condition;

FIG. 4A illustrates the decrease in contact angel with increased surface energy or decreased surface tension;

FIG. 5 is another view similar to FIGS. 2A and 2C illustrating the electromagnetic radiation going through the filter material;

FIG. 5A is another view similar to FIG. 5 but showing the electromagnetic radiation traveling in a direction that is co-linear or generally parallel with the direction of the airstream;

FIG. 6-6A are also views that illustrates different positionings of the electromagnetic generators;

FIG. 7 is a view showing an electromagnetic radiation generator adjacent to filter material and showing the electromagnetic radiation traveling therethrough;

FIG. 8 is a view of another embodiment illustrating electromagnetic radiation traveling through a circular filter;

FIG. 9 is a view illustrating an electromagnetic generator situated inside a filter, with radiation traveling through the filter; and

FIG. 10 is a schematic view illustrating a filter volume that defines the volume of the filter material in the filter.

### DETAILED DESCRIPTION OF THE PREFERRED EMBODIMENTS

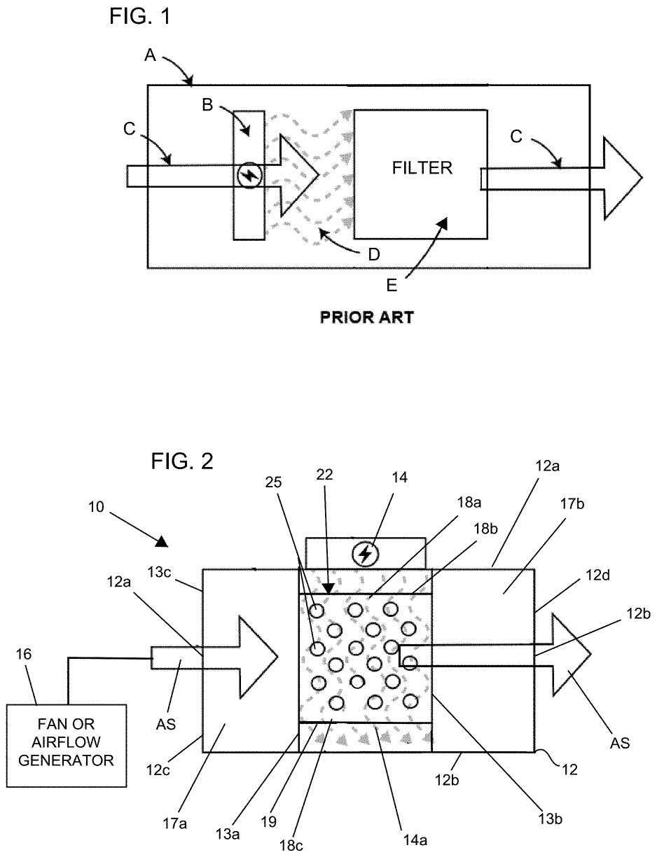

FIG. 1 illustrates a view of a prior art system showing an air housing A comprising an internal electromagnetic generator B, an air outflow or outlet and an air inflow C, a non-penetrating radiation D, and a non-transmissible filter material E. As air flows through the prior art housing A, the electromagnetic generator B generates a non-penetrating radiation D that is applied to the airstream to decontaminate it. During a second operation, the non-transmissible filter material E mechanically filters the airstream after the radiation is applied.

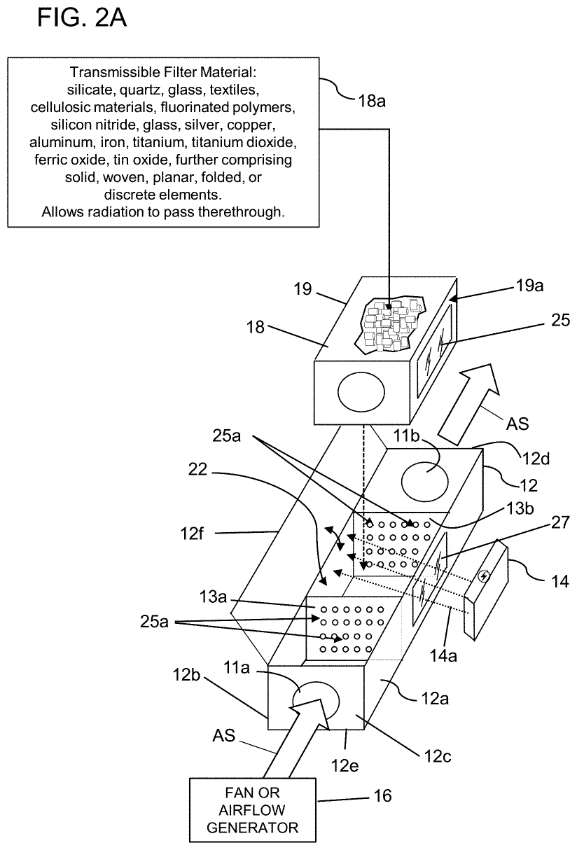

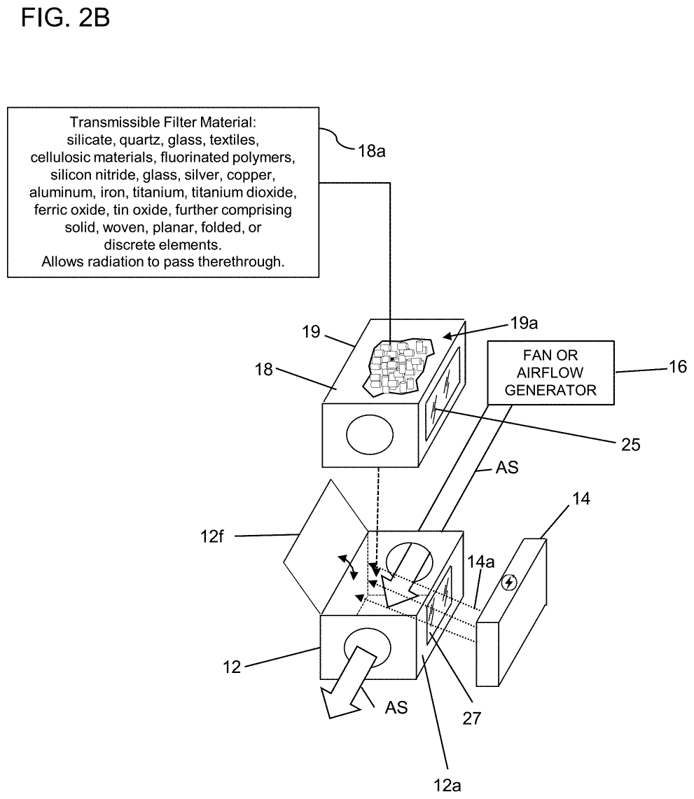

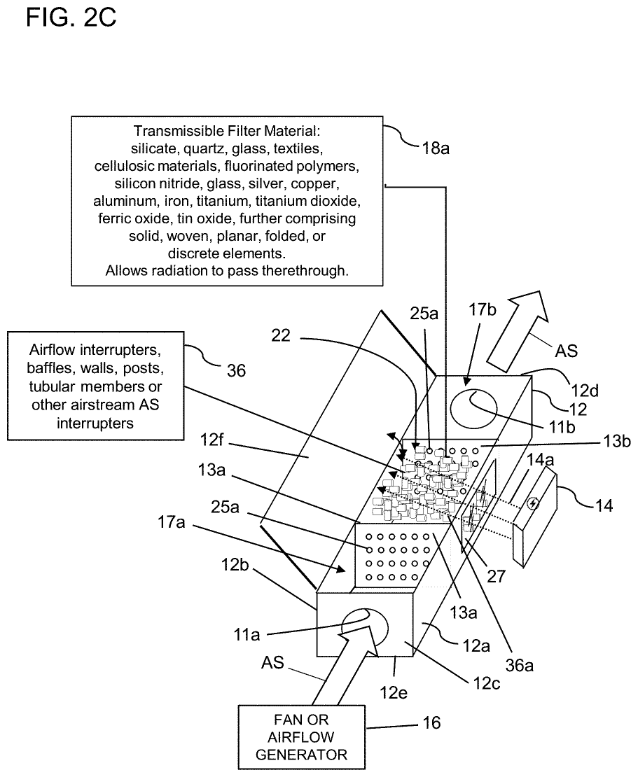

An improved and more efficient decontamination system 10 will now be described relative to FIGS. 2-10. The decontamination system 10 comprises a housing 12 that houses or supports an electromagnetic generator 14 and at least one transmissible filter 18. The electromagnetic generator 14 generates irradiation energy 14a effective to irradiate and decontaminate an airstream AS passing through the housing 12 and through at least one transmissible filter 18 as will be described herein. This embodiment is ideally energy-based, comprising the electromagnetic spectrum including ultraviolet, infrared, electrostatic, and/or irradiation means. The contaminants can include pathogenic bacteria, contaminated bioaerosols, fungi, spores, or viruses. In a hospital or medical center application the decontamination reduces the risk of hospital acquired infections, especially surgical site infections, in patients.

In the illustration being described, the decontamination system 10 could comprise or be situated in an ILLUVIA® decontamination air handler product available from Aerobiotix, Inc. of Miamisburg, Ohio. In the illustration being described, one suitable decontamination system 10 may include the system, apparatus or features of the air handler and irradiation devices shown in U.S. Pat. Nos. 9,433,693; 9,457,119; 9,764,054; 10,039,854; 10,532,122 and 10,549,007; as well as U.S. Patent Publication Nos. 2018/0133084; 2018/0133355; 2018/0264391; 2019/0099050 and 2020/0047094, all of which are assigned to the same assignee as the present application and are incorporated herein by reference and made a part hereof.

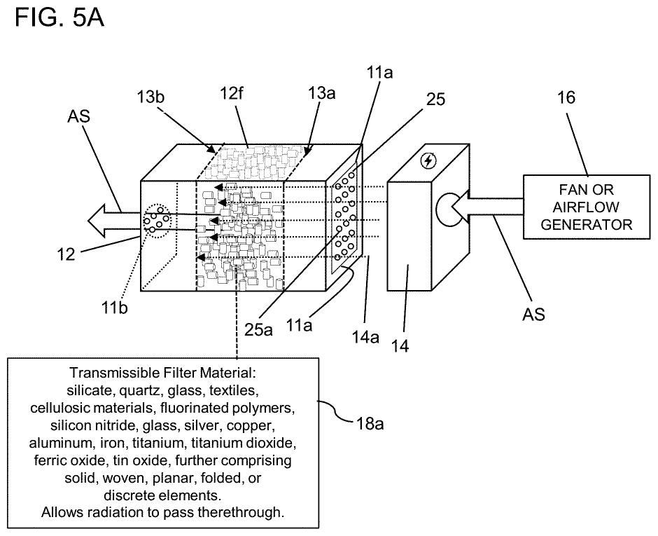

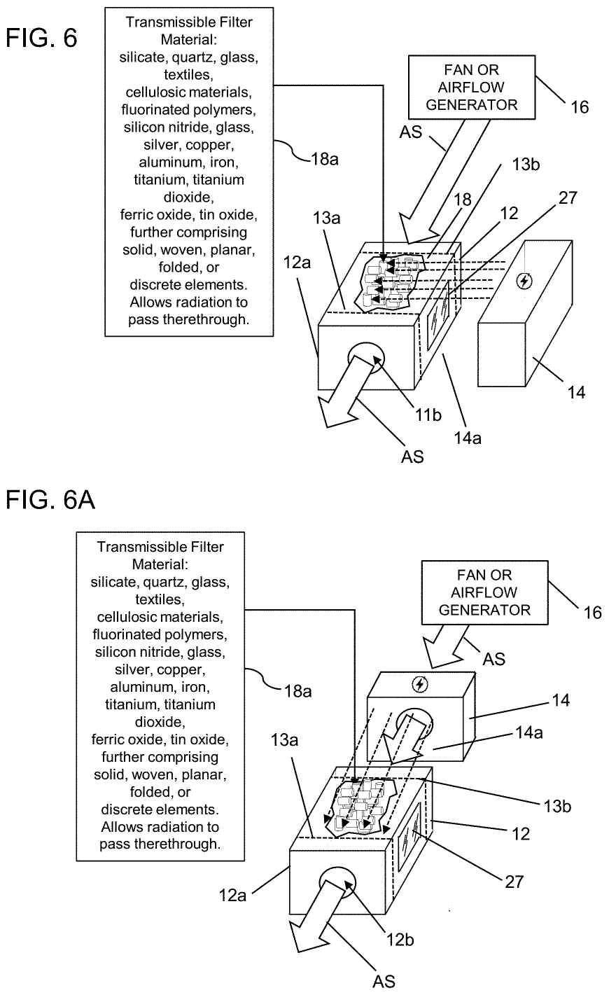

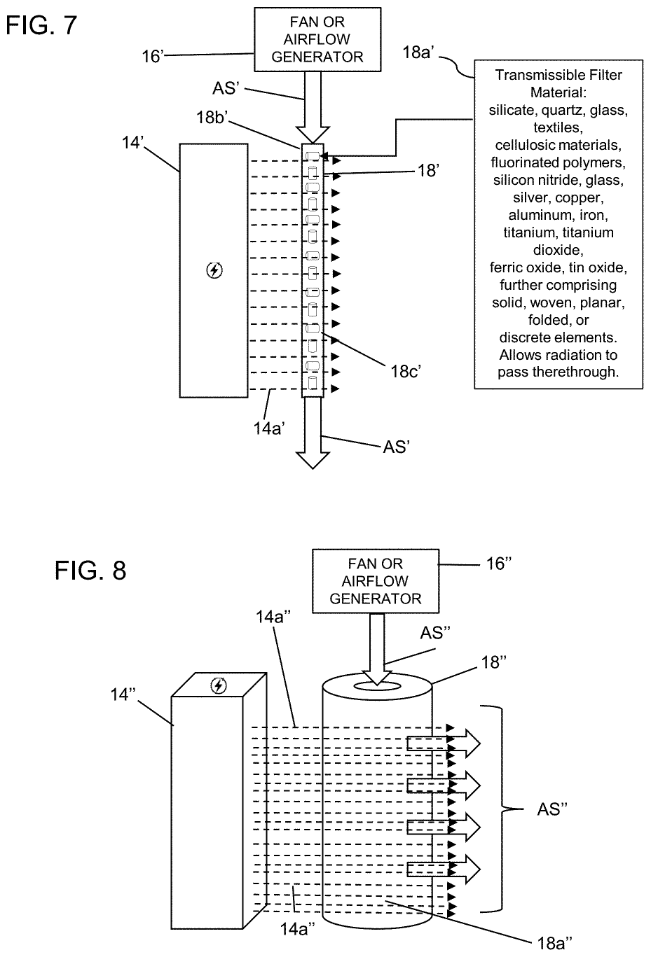

FIGS. 2-2C illustrate various features of one embodiment of the invention, which will now be described. The decontamination system 10 comprises the housing 12 (FIGS. 2-2C, 5, 5A and 6) having congruous walls 12a-12d, a bottom wall 12e and a top wall or surface 12f, which is not shown in FIG. 2 for ease of illustration. The top wall or surface 12f may be fixed or secured to walls 12a-12d as shown in FIG. 5A or, alternatively, it could be hingeably mounted to the wall 12b (FIG. 2C) and moveable from an open position shown in FIG. 2C to a closed position (illustrated in FIGS. 5, 5A, and 6-6A). The walls 12a-12d define a generally rectangular filter receiving area 22 (FIG. 5A) adapted and sized to receive the at least one transmissible filter 18, as illustrated in FIGS. 2-2A. It is important to note that the at least one transmissible filter 18 is comprised of filter material 18a that may be housed in a frame 19 (FIGS. 2, 2A and 2B) or the filter material 18a could be frameless (FIG. 7). Alternatively, the filter material 18a may be loosely and/or randomly arranged in the filter receiving area 22 as illustrated in FIG. 2C.

Thus, the system 10 could be standalone or integrated with a freestanding housing or air handler (not shown) of the type shown and described in the aforementioned patents. The air handler may be adapted to have walls that define the walls 12a-12f of the housing 12. As shown, the housing 12 can be free-standing, be integrated with an air handler or decontamination system, or be housed or supported in a duct in a larger system. The walls (not shown) of the air handler or duct may provide and define the housing 12 if desired. Thus, the decontamination system 10 could be situated or arranged in a housing 12 in the form of a duct, for example, such as a duct in an HVAC system or other system that handles or directs air. Alternatively, the housing 12 could stand-alone and be placed in a desired location where it can receive an airstream AS to decontaminate.

As illustrated in FIGS. 2 and 2A, the system 10 may comprise the substantially congruous walls 12a-12d in a monolithic, one-piece, welded or molded construction. Note that the housing 12 walls 12a and 12b are coupled to internal walls 13a and 13b that are generally opposed as shown. These walls 13a, 13b, 12a, 12b and bottom wall 12e and top wall 12d cooperate to define the filter receiving area 22 that is adapted to receive the at least one transmissible filter 18. The housing 12 comprises an end wall portion 12d that generally opposes wall 13b to define a pre-chamber area 17a and wall 12c being generally opposed to wall 13a to define a post-chamber area 17b as illustrated. The housing 12 further comprises the bottom wall 12e and a generally opposed top wall 12f which can be fixed or hingeable as mentioned earlier. For ease of illustration and description, the top wall 12f is removed in the FIGS. 2 and 2A and is shown hingeably mounted to the wall 12b (FIG. 2C) in a conventional manner so that the inside of the housing 12 and filter receiving area 15 may be accessed by opening the top wall 12f as shown. Thus, the top wall 12d is either integrally formed and fixed (FIG. 5) or is shown as being hingeable or removable as illustrated in FIG. 5A. It should be understood that any of the walls 12a-12f could be adapted to open or removed if desired.

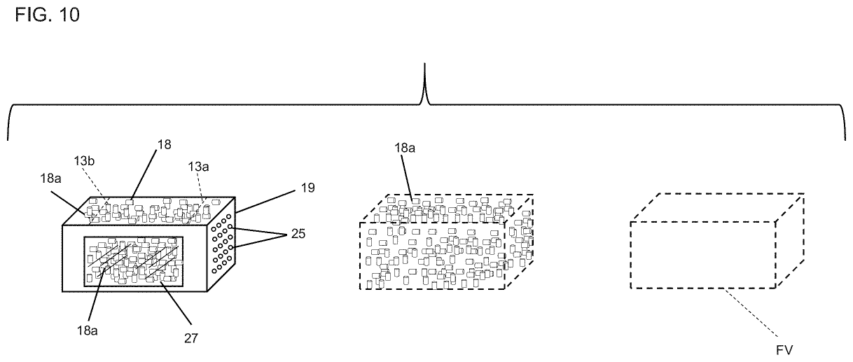

In one embodiment, the walls 13a and 13b comprise at least one opening, aperture or fenestrations 25a to permit the airstream AS to pass through the housing 12. The wall 12a (FIG. 2C) has at least one operative opening 11a that is operatively associated with the electromagnetic generator 14 to permit the electromagnetic radiation 14a to pass into the filter material 18a in the filter receiving area 22 such that the electromagnetic radiation 14a fills the entire filter receiving area 22 and filter material 18a when the electromagnetic generator 14 is energized. Alternatively, it should be understood that one or more of the walls 12a-12f or a portion thereof could be non-opaque or could comprise a transparent window 27 (FIGS. 2B and 10) so that the electromagnetic generator 14 can be operatively associated therewith so that the electromagnetic radiation 14a may pass through the transparent window 27 and into the filter receiving area 22 as illustrated. In the illustration, the electromagnetic generator 14 is shown spaced from the at least one transmissible filter 18 for ease of illustration, but preferably, the electromagnetic generator 14 is situated adjacent to or against the at least on transmissible filter 18. Thus, as illustrated in FIG. 6, note that the wall 12a is solid but comprises the transparent window 27 to permit the electromagnetic radiation 14a to be transferred into the filter receiving area 22 where the filter material 18a resides. In the illustrations being described, it should be understood that electromagnetic radiation does not escape or get transmitted outside of the filter receiving area 22 of housing 12.

As mentioned earlier, the components of the electromagnetic generator 14 and at least one transmissible filter 18 may be housed in the filter receiving area 22, such as an air handler housing or a housing defined by a duct in an HVAC system. It is important to note that the filter receiving area 22 and the electromagnetic generator 14 are situated adjacent to or in operative relationship with the at least one transmissible filter 18 and filter receiving area 22 in whatever environment they are placed so that the electromagnetic generator 14 can generate electromagnetic energy 14a that is transmitted into the filter receiving area 22 and into and through the at least one transmissible filter 18 and filter material 18a. In this regard, note that the filter material 18a defines a predetermined filter volume FV that is illustrated in FIG. 10. This FIG. 10 shows the at least one transmissible filter 18′″ that comprises a frame 19′″ and the filter material 18a′″ of the decontamination system 10′″ (left side of the figure). The middle portion of FIG. 10 shows the filter material 18a′″ removed from the frame 19′″ and to the right of that FIG. 10 is a view illustrating in phantom a predetermined filter volume FV′″ that is filtered by the electromagnetic radiation 14a′″ generated by the electromagnetic generator 14′″.

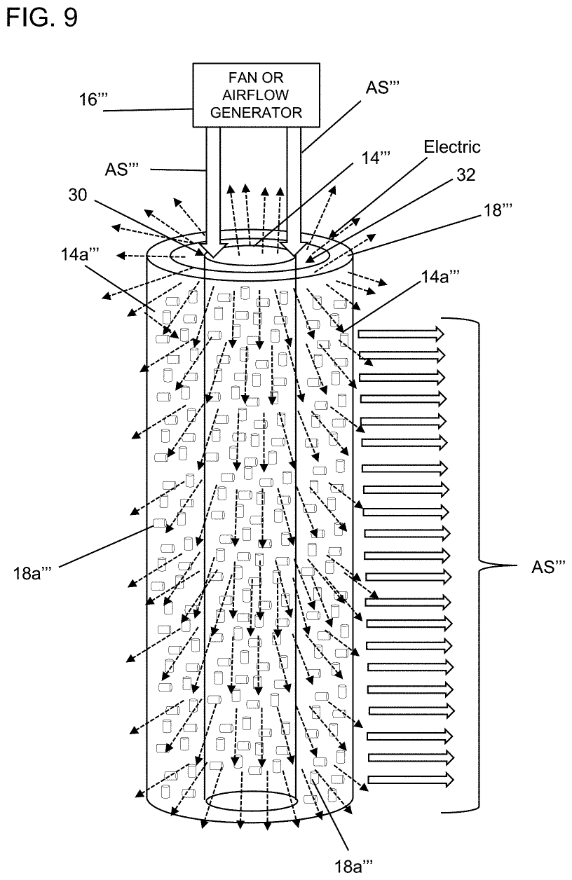

Thus, the electromagnetic generator 14 is preferably set adjacent or juxtaposed to the at least one transmissible filter 18 (as illustrated in FIGS. 2-2C, 7 and 8). Other arrangements in FIGS. 7-9 are contemplated in which like parts have been indicated by a prime mark(s) “′” as shown. FIG. 7 shows the electromagnetic generator 14′ situated adjacent to the at least one filter 18′ and applies radiation 14a′ generally perpendicular to the airstream AS′. FIG. 8 shows an embodiment where FIG. 9 shows the electromagnetic generator 14′″ in the form of a UV lamp situated internally and/or at least partially or completely surrounded by the at least one transmissible filter 18′″. In one embodiment, the electromagnetic generator 14′″ comprises at least one or a plurality of UV (ultraviolet) lamps 30 (only one is shown in FIG. 9) that is situated in an area 32 defined by the circular or tubular filter 18′″ and filter material 18a′″.

As mentioned earlier, the at least one transmissible filter 18 comprises the filter material 18a that will be described in more detail later herein. The at least one transmissible filer 18 and electromagnetic generator 14 are separate components, but may be contained in the congruous outer housing 12, as mentioned. The only special aspect or feature is that electromagnetic generator 14 releases radiation energy in a sufficient quantity to penetrate the filter material 18a and transmit through, and create an energy field density high enough to obtain microbial killing in the airstream AS passing therethrough. The electromagnetic radiation 14a generated by the electromagnetic generator 14 fills the entire predetermined filter volume FV or filter receiving area 22 to substantially simultaneously irradiate the airstream AS while the airstream AS is passing through the at least one transmissible filter 18 and being mechanically filtered by the at least one transmissible filter 18 and/or the filter material 18a.

The decontamination system 10 may comprise a fan or airflow generator 16 (FIG. 2) for generating or causing the airstream AS to flow into an inlet 11a (FIG. 2A), pass through the housing 12 and the at least one transmissible filter 18, and then through an outlet 11b of the housing 12 where the disinfected and filtered airstream AS can pass into the local environment, such a surgery room, hospital room, building, home or other room or area of a structure. Alternatively, the decontamination system 10 may rely on airflow from a separate air handler (not shown), such as the air handler in an HVAC system in a building that generates the airstream AS.

It is important to note that the decontamination system 10 comprises the at least one transmissible filter 18 for filtering the airstream AS and also for cooperating with the electromagnetic generator 14 to cause contaminants and bioaerosols to be irradiated when the airstream AS passes through the at least one transmissible filter 18 and the housing 12. Note that, unlike the prior art system shown in FIG. 1, the at least one transmissible filter 18 and the electromagnetic generator 14 in the embodiment shown in FIG. 2 are juxtaposed or situated adjacent to and in operative relationship with each other as mentioned earlier. FIGS. 2-6A show the electromagnetic generator 14 slightly spaced from the wall 12a for ease of illustration of the various components. It is important to understand that the filter material 18a permits electromagnetic radiation to pass or permeate therethrough so that the at least one transmissible filter 18 mechanically filters the airstream AS as the electromagnetic generator 14 generates electromagnetic radiation that passes through the filter material 18a to substantially simultaneously irradiate the airstream AS as it is being filtered by the at least one transmissible filter 18.

Thus, it should be understood that the filter material 18a and airstream AS are both substantially simultaneously irradiated by the electromagnetic generator 14 to disinfect and decontaminate the airstream AS as it is being filtered by the at least one transmissible filter 18. One feature of this embodiment is that because the filter material 18a is being continuously radiated, it reduces or eliminates altogether the need to replace it because it is being constantly “cleaned” by the irradiation.

The electromagnetic generator 14 is adapted to irradiate, disinfect and/or decontaminate the airstream AS as it flows past the electromagnetic generator 14 and through the at least one transmissible filter 18. In this regard, the electromagnetic generator 14 generates the penetrating electromagnetic wave and radiation 14a in a sufficient quantity to penetrate the filter material 18a of the at least one transmissible filter 18. The energy field density of the radiation from the electromagnetic generator 14 is sufficient in energy, wavelength and frequency to create an energy field density that is high enough to obtain microbial killing. It is also important to note that the radiation penetrates the entire predetermined filter volume FV (FIG. 10) defined by the filter material 18a of the at least one transmissible filter 18 and not only decontaminates the filter material 18a by killing unwanted pathogens and unwanted microbial contamination that are resident on the filter material 18a, but also that are located in proximity to a first side 18b (FIG. 7) and a second side 18c as the airstream AS flows through and past the at least one transmissible filter 18.

Details of the at least one transmissible filter 18, filter material 18a and the electromagnetic radiation 14a will now be described relative to FIGS. 2-9. The electromagnetic generator 14 is situated adjacent or juxtaposed to the at least one transmissible filter 18 as mentioned earlier. The electromagnetic generator 14 generates electromagnetic radiation 14a of sufficient waveform, force and energy to fill the entire predetermined filter volume FV of the at least one transmissible filter 18 and the filter material 18a with electromagnetic radiation suitable to decontaminate the airstream AS passing therethrough. It is important to understand that this results in substantially simultaneous electromagnetic radiation and decontamination by the electromagnetic generator 14 and mechanical filtration by the filter material 18a. In one embodiment illustrated in FIG. 7, notice that the radiation not only fills the predetermined filter volume FV defined by the at least one transmissible filter 18, but passes through the at least one transmissible filter 18. Notice also that the electromagnetic generator 14 may be situated such that its radiation is generally perpendicular or orthogonal to the airstream AS direction of flow as shown in FIGS. 2-7. Alternatively, the electromagnetic radiation 14a may flow in a direction that is co-linear with or parallel to the direction of flow of the airstream AS as mentioned earlier and as illustrated in FIGS. 5A, 6A and 7-9. Thus, in a preferred embodiment, the electromagnetic radiation penetrates and passes through the entire at least one transmissible filter 18 and the filter material 18a.

Advantageously, the electromagnetic radiation 14a permeates the entire predetermined filter volume FV (illustrated in phantom on the right side of FIG. 10) of the filter material 18a such that the electromagnetic radiation is detectable throughout substantially all of the predetermined filter volume FV, thereby creating an electromagnetic field substantially filling or traversing the predetermined filter volume FV. It is also important to note that the filter material 18a is substantially transmissible to the electromagnetic energy generated by the electromagnetic generator 14 so the electromagnetic radiation can pass therethrough

Referring to FIGS. 5A and 10, notice that the at least one transmissible filter 18 comprises the filter material 18a that defines the predetermined filter volume FV (FIG. 10), which is schematically shown in phantom in FIG. 10. FIG. 2A illustrates that the filter material 18a may be provided with the frame 19 or without the frame 19 (FIGS. 2A-2C, 6A and 10). Preferably, if the at least one transmissible filter 18 comprises the frame 19, then the frame 19 may be designed to frame the filter material 18a such that some sides are closed, while at least one wall or side 12a (FIG. 2B) has the aperture or transparent window 27 or the like to permit electromagnetic radiation 14a to pass into and through the filter material 18a. In order to enhance electromagnetic radiation of the airstream AS within the at least one transmissible filter 18 and/or to increase a dwell time during which the airstream AS is subjected to the radiation energy 14a from the electromagnetic generator 14, the interior surfaces of the filter frame 19 (FIG. 2A) may be mirrored, colored or adapted to internally reflect the electromagnetic radiation. Alternatively, the interior surfaces of the walls 12a-12f that define the housing 12 may be adapted similarly to facilitate directing or maintaining the electromagnetic radiation 14a within the filter receiving area 22 and predetermined filter volume FV of the at least one transmissible filter 18. It should be understood that in one embodiment, no radiation leaves the housing 12 during operation.

In the illustration being described, the electromagnetic radiation 14a comprises an ultraviolet or near ultraviolet spectrum between 10-420 nm wavelength. The predetermined filter volume FV (FIG. 10) comprises or is defined by the filter material 18a that is substantially transmissible to this spectrum. The electromagnetic radiation may also comprise non-ionizing radiation, ionizing radiation, ultraviolet, infrared, electrons, electrostatics, plasma, light, laser, LED, lamp, excited gas, and similar phenomena which function to effect biological and non-biological contamination within the airstream AS.

As mentioned earlier, the filter material 18a could also be framed with the frame 19 (FIG. 2A) or the filter material 18a may be frameless as shown in FIG. 7. FIG. 7 shows the electromagnetic generator 14 situated adjacent to the filter material 18a without a frame 19. These components may be housed in a separate housing (not shown), other than the housing 12. Alternatively and as mentioned earlier herein, an air handler or duct (not shown) may provide one or more of the walls 12a-12f that surround and house these components as shown in FIG. 5A. Notice that the filter material 18a may be randomly distributed in the filter receiving area 22 as illustrated in FIGS. 2C, 5A and 6-6A. In these examples, the filter material 18a is loose and poured or randomly situated in the filter receiving area 22. Note that the wall 12c (FIG. 5C) is fenestrated with at least one or a plurality of apertures or fenestrations 25a to permit electromagnetic radiation 14a to pass into the filter receiving area 22 and through the filter material 18a.

Advantageously, the decontamination system 10 is adapted to and comprises means to increase dwell time of the airstream AS within the at least one transmissible filter 18 and the filter material 18a. Because the electromagnetic energy traverses the entire predetermined filter volume FV, the increased dwell time that the airstream AS is subjected to radiation is improved which improves the effectiveness of the mechanical filtration and electromagnetic radiation. The filter receiving area 22 (FIG. 2C) of the housing 12 or the filter material 18a may have at least one baffle or airflow interrupter 36 (FIG. 2C), baffles, walls, posts, disrupters or tubular members, for example, to facilitate disrupting the airstream AS in order to increase dwell time. As mentioned earlier, one or more features of the patents referenced earlier herein may be incorporated and used with one of more of the embodiments described herein. In this regard, one or more airflow interrupters 36 may be placed inside the filter 18 or in the receiving area 22 in the housing 12 of the decontamination system 10 to interrupt the airstream AS and increase dwell time in the filter receiving area 22 so that the airstream AS is subjected to the UV radiation for a longer period. For example, one or more small tubular members or quartz members may be situated in the decontamination system 10 as shown. Thus, the reflective, focusing, concentrating or baffling apparatus or airflow interrupter 36 may create turbulence within the air stream AS passing through the housing 12 in order to improve the ultraviolet or radiation effectiveness. For example, features of the system and devices of U.S. Pat. No. 9,457,119 issued Oct. 4, 2016, may be used. This patent is owned by the same Assignee as the present application and is incorporated herein by reference and made a part hereof as mentioned earlier. Again, such features may include providing at least one baffle or other airflow interrupter 36, such as the at least one baffle or airflow interrupter 36 that may comprise a plurality of tubular quartz members 36a (FIG. 2C) that are situated in the decontamination system 10.

It is important to note that the filter material 18a itself comprises materials or means for facilitating increasing dwell time and or that defines a surface 18a1 (FIG. 7) that functions to aerodynamically entrap or capture air contaminates such that the contaminants are transiently or permanently captured within the filter material 18a of the at least one transmissible filter 18. This, in turn, increases dwell time or exposure to the electromagnetic radiation 14a within the predetermined filter volume FV defined by the filter material 18a.

As previously mentioned, the at least one transmissible filter 18 comprises the filter material 18a, which may be frameless, housed in the filter body or frame 19 or even loosely arranged in the filter receiving area 22 (FIG. 2C). The filter material 18a comprises at least one of the following materials alone or in combination: silicate, quartz, glass, textiles, cellulosic materials, fluorinated polymers, silicon nitride, glass, silver, copper, aluminum, iron, titanium, titanium dioxide, ferric oxide, tin oxide, further comprising solid, woven, planar, folded, or discrete elements. One important aspect of one embodiment being described is that the electromagnetic radiation is detectable through substantially all of the at least one transmissible filter 18, filter material 18a and predetermined filter volume FV (FIG. 7) during use and as the at least one transmissible filter 18 substantially simultaneously mechanically filters the airstream AS.

In the illustration, the filter material 18a may be any suitable material which can transmit or permit transmission of radiation, such as electromagnetic radiation 10-420 nm wavelength or ultraviolet or near ultraviolet wavelength, but it should be understood that other levels, type and forms of radiation may be used. For example, quartz can transmit ultraviolet, and low density materials like polymers or cellulosic materials can transmit microwave radiation, and the like. The filter material 18a can consist of multiple discrete units, or comprise a continuous membrane. In the preferred embodiment, it has a hydrophilic surface for attracting aerosols.

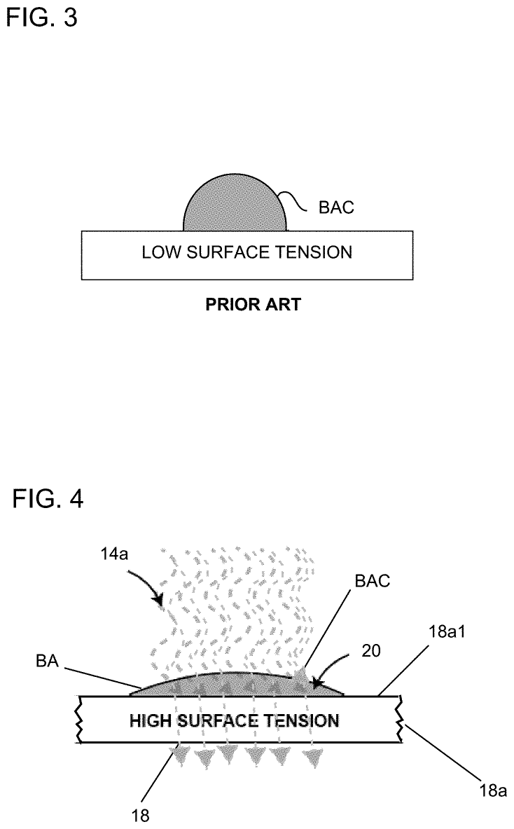

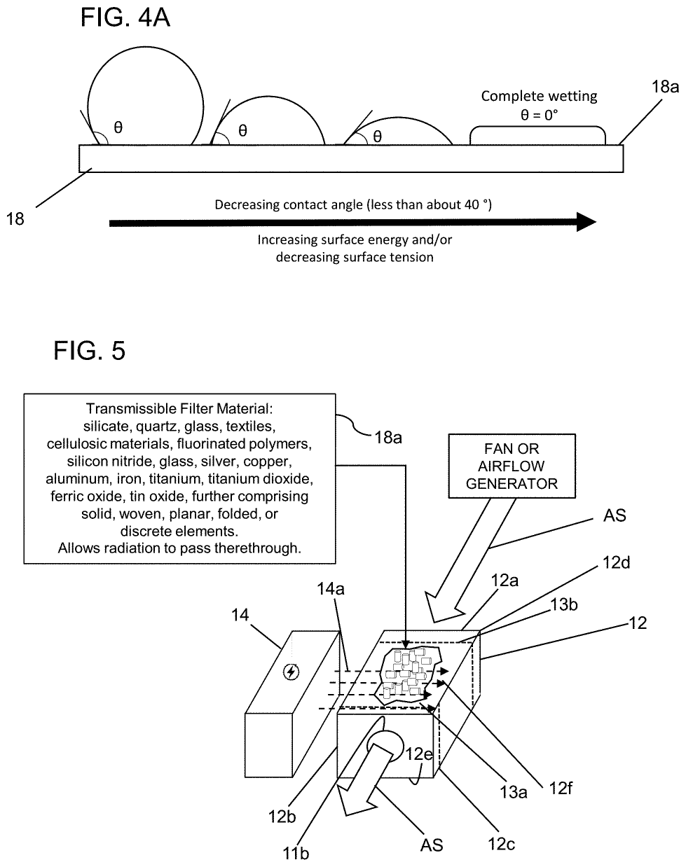

An improved means for maximizing interaction between the electromagnetic radiation energy 14a and the bioaerosol contaminants that adhere to the filter material 18a and this will now be described relative to FIGS. 3, 4 and 4A. Referring now to FIG. 4A, a water or bioaerosol contact angle θ is shown. This angle θ is a correlation or measure of hydrophilicity. It should be understood and as mentioned herein, the filter material 18a may comprise or take any form, as long as it achieves the required surface tension, also known as surface energy, wicking or wettability. It can be a function of the surface material 18a itself or any material processed to increase surface energy or chemical bonding activity. Such processes may comprise one or more of the following processes: plasma treatment, corona treatment, chemical coating, photocatalytic treatment, deposition, oxidation, and/or etching. For example, the surface 18a1 could comprise a photocatalytic coating or surface that facilitates hydrophilicity and wettability.

The filter material surface 18a of the embodiments being described is illustrated in FIGS. 4-4A, with a comparative view of the prior art in FIG. 3. A bioaerosol contaminant BAC (FIGS. 3 and 4) in the form of a bioaerosol droplet having an undesired contaminant is shown in a low surface tension condition. FIG. 4A illustrates the water or bioaerosol contact angle WCA of less than about 40 degrees in one embodiment. Note that because of the low surface tension between the bioaerosol BAC and the prior art material surface (FIG. 3), the bioaerosol BAC does not wet out and remains relatively uniform in a droplet shape as shown in FIG. 3. This actually minimizes interaction between the bioaerosol and the electromagnetic radiation.

In contrast, by selecting a filter material 18a composition or by applying a wetting, coating, treatment or other processes mentioned herein to the filter material 18a itself or filter material surface, such as surface 18a1 (FIGS. 4 and 4A), the wetting and wicking out can be maximized. This, in turn, leads to improved exposure or dwell time of the bioaerosol droplet BAC to the electromagnetic radiation 14a. This improves overall decontamination of the airstream AS by the system 10. Note in the illustration in FIGS. 4-4A that the bioaerosol BA wicks or wets out and is in a high surface tension condition which makes it spread out. This maximizes interaction between the electromagnetic radiation 14a and the bioaerosol BA contaminant.

### Additional Considerations

1. Advantageously, one potential benefit of this decontamination system 10 is that replacement of the at least one transmissible filter 18 may be less frequent or not even needed because the at least one transmissible filter 18 and filter material 18a are being continuously “cleaned” or exposed to radiation, at least for biological contaminant. This saves a user from continuously having to purchase and replace the at least one transmissible filter 18. This can result in huge savings to the user, especially with widespread use of the system 10, such as in an environment where the system 10 is located in each room of a building, such as patients and surgery rooms in a medical facility.

2. In the illustration, it is important to note that the housing 12 and walls and surfaces 12a-12f prevent escape of radiation into the environment and it has the air inlet 12a and air outlet 12b.

3. As mentioned earlier, the housing 12 may be adapted such that at least one wall or surface 12a-12f that is situated adjacent to or in operative relationship with the electromagnetic generator 14. As described earlier relative to FIGS. 2A and 2C, the at least one transmissible filter 18 comprise the aperture 25 (FIG. 2C) that becomes generally associated with the transparent window 27 (FIG. 2B) of the housing 12 to permit electromagnetic radiation 14a to pass into the filter material 18a. Thus, the wall 12a and window 25 of the at least one transmissible filter 18 or a portion thereof may be situated adjacent to the electromagnetic generator 14 may be transparent so that the electromagnetic waveforms can pass therethrough and into the filter material 18a. The window 25 of the at least one transmissible filter 18 may be open or may comprise a transparent window 25a of the filter material 18a loosely situated in the at least one transmissible filter 18.

4. FIG. 7 shows an embodiment where the electromagnetic generator 14 is situated directly adjacent the filter material 18a and is not separated by any wall at all. As mentioned earlier herein, these two components may be placed directly into an environment where they are housed, such as in an air handler or a duct, or could be provided in the separate housing 12 or an air handler (not shown) of the type shown and described herein. As mentioned earlier, the filter material 18a may be provided with the frame 19 (FIG. 2A) or without a frame as illustrated in FIG. 7. As mentioned earlier, the filter material 18a could be loose and loosely arranged in the filter receiving area 22 of the housing 12.

5. In the illustration, the filter material 18a is transmissible which means radiation can pass therethrough. However, the filter material 18a itself may be non-transmissible, yet arranged, adapted or constructed to permit electromagnetic radiation 14a to pass through the at least one transmissible filter 18, while the filter material 18a is exposed to electromagnetic radiation 14a, even if radiation does not pass through the filter material 18a itself.

6. It should be understood that the filter material 18a itself may be opaque, but arranged with spacing such that electromagnetic radiation 14a may pass through the at least one transmissible filter 18.

This invention, including all embodiments shown and described herein, could be used alone or together and/or in combination with one or more of the features covered by one or more of the claims set forth herein, including but not limited to one or more of the features or steps mentioned in the Summary of the Invention and the claims.

While the system, apparatus and method herein described constitute preferred embodiments of this invention, it is to be understood that the invention is not limited to this precise system, apparatus and method, and that changes may be made therein without departing from the scope of the invention which is defined in the appended claims.

What is claimed is:

1. An energy-transmissible air filtration system comprising:
at least one transmissible filter comprising a filter material that is adapted to permit a substantially transmissible application of decontamination energy adapted to decontaminate an airstream having contaminants flowing though the at least one transmissible filter;
wherein said decontamination energy traverses an interior area of said filter in order to apply said decontamination energy substantially simultaneously as said filter material mechanically filters said airstream as said decontamination energy is applied.

2. The energy-transmissible air filtration system as recited in claim 1 wherein said filter material is adapted to at least one of aerodynamically influence said contaminants in said airstream to entrap contaminants such that said contaminants are transiently or permanently captured within said filter material.

3. The energy-transmissible air filtration system as recited in claim 2 wherein at least one transmissible filter comprises:
a filter body having an exterior surface, said filter body defining said interior area to have a predetermined volume;
said filter material permitting said decontamination energy to be substantially transmissible in order to permeate said interior area and said predetermined volume.

4. The energy-transmissible air filtration system as recited in claim 1 wherein said filter material comprises at least one of silicate, quartz, glass, textiles, cellulosic materials, fluorinated polymers, silicon nitride, glass, silver, copper, aluminum, iron, titanium, titanium dioxide, ferric oxide, tin oxide, further comprising solid, woven, planar, folded, or discrete elements.

5. The energy-transmissible air filtration system as recited in claim 1 wherein said decontamination energy comprises at least one of: a non-ionizing radiation, an ionizing radiation, an ultraviolet, an infrared, electrons, electrostatics, a plasma, a light, a laser, an LED, a lamp, an excited gas, and similar phenomena which function to effect biological and non-biological contamination within said airstream.

6. The energy-transmissible air filtration system as recited in claim 3 wherein said decontamination energy is generated by a decontamination energy generator that generates said decontamination energy such that it substantially fills or traverses said predetermined volume.

7. The energy-transmissible air filtration system as recited in claim 1 wherein said decontamination energy is generated by a decontamination energy generator that generates said decontamination energy such that it substantially fills or traverses said predetermined volume.

8. The energy-transmissible air filtration system as recited in claim 1 wherein said decontamination energy is applied by a decontamination energy generator, said decontamination energy generator generates ultraviolet or near ultraviolet spectrum between 10-420 nm wavelength.

9. The energy-transmissible air filtration system as recited in claim 8 wherein at least one of said filter material or said internal area comprises a material that permits said spectrum to be substantially transmissible therethrough.

10. The energy-transmissible air filtration system as recited in claim 1 wherein filter material comprises a hydrophilic surface with a water contact angle of less than about 40 degrees.

11. The energy-transmissible air filtration system as recited in claim 1 wherein filter material comprises a hydrophilic surface having a surface tension greater than 40 dynes/cm in order to facilitate increasing at least one of a surface-bioaerosol interaction or an exposure or dwell time within said electromagnetic field.

12. The energy-transmissible air filtration system as recited in claim 10 wherein filter material comprises a hydrophilic surface having a surface tension greater than 40 dynes/cm in order to facilitate increasing at least one of a surface-bioaerosol interaction or an exposure or dwell time within said electromagnetic field.

13. An energy-transmissible air filtration system comprising:
a housing having an inlet and an outlet adapted to permit an airstream to pass therethrough;
an decontamination energy generator for generating electromagnetic energy effective to remove at least a portion of biological contaminants in said airstream; and
at least one transmissible filter having a transmissible filter material adapted to permit said electromagnetic waves to substantially pass therethrough, said at least one transmissible filter being defining an internal area having a predetermined volume;
said decontamination energy generator being situated in operative relationship with said at least one transmissible filter is substantially transmissible such that said electromagnetic energy may pass therethrough while said decontamination energy generator substantially simultaneously said decontamination energy generator applies said electromagnetic energy to said airstream as it passes through said filter.

14. The energy-transmissible air filtration system as recited in claim 13 wherein said transmissible filter material is adapted to permit a substantially transmissible application of electromagnetic energy sufficient to substantially decontaminate said airstream having contaminants flowing though the filter as said filter material mechanically filters said airstream.

15. The energy-transmissible air filtration system as recited in claim 13 wherein said filter material is adapted to at least one of aerodynamically influence said contaminants in said airstream of entrap contaminants such that said contaminants are transiently or permanently captured within said filter material.

16. The energy-transmissible air filtration system as recited in claim 14 wherein at least one transmissible filter comprises:
a filter body having an exterior surface, said filter body defining said interior area to have a predetermined volume;
said filter material permitting said decontamination energy to be substantially transmissible in order to permeate said interior area and said predetermined volume.

17. The energy-transmissible air filtration system as recited in claim 13 wherein said filter material comprises at least one of: silicate, quartz, glass, textiles, cellulosic materials, fluorinated polymers, silicon nitride, glass, silver, copper, aluminum, iron, titanium, titanium dioxide, ferric oxide, tin oxide, further comprising solid, woven, planar, folded, or discrete elements.

18. The energy-transmissible air filtration system as recited in claim 13 wherein said decontamination energy comprises at least one of: a non-ionizing radiation, an ionizing radiation, an ultraviolet, an infrared, electrons, electrostatics, a plasma, a light, a laser, an LED, a lamp, an excited gas, and similar phenomena which function to effect biological and non-biological contamination within said airstream.

19. The energy-transmissible air filtration system as recited in claim 15 wherein said decontamination energy is generated by a decontamination energy generator that generates said decontamination energy such that it substantially fills or traverses said predetermined volume.

20. The energy-transmissible air filtration system as recited in claim 13 wherein said decontamination energy is generated by a decontamination energy generator that generates said decontamination energy such that it substantially fills or traverses said predetermined volume.

21. The energy-transmissible air filtration system as recited in claim 13 wherein said decontamination energy is applied by a decontamination energy generator, said decontamination energy generator generates ultraviolet or near ultraviolet spectrum between 10-420 nm wavelength.

22. The energy-transmissible air filtration system as recited in claim 21 wherein at least one of said filter material or said internal area comprises a material that permits said spectrum to be substantially transmissible therethrough.

23. The energy-transmissible air filtration system as recited in claim 13 wherein filter material comprises a hydrophilic surface with a water contact angle of less than about 40 degrees.

24. The energy-transmissible air filtration system as recited in claim 13 wherein filter material comprises a hydrophilic surface having a surface tension greater than 40 dynes/cm in order to facilitate increasing at least one of a surface-bioaerosol interaction or an exposure or dwell time within said electromagnetic field.

25. The energy-transmissible air filtration system as recited in claim 23 wherein filter material comprises a hydrophilic surface having a surface tension greater than 40 dynes/cm in order to facilitate increasing at least one of a surface-bioaerosol interaction or an exposure or dwell time within said electromagnetic field.

26. An energy-transmissible filter comprising:
a filter housing; and
a filter material located in said housing;
said filter material permitting electromagnetic energy to traverse therethrough to facilitate decontaminating an airstream passing through said filter as said filer material mechanically filters said filter material.

27. The energy-transmissible filter as recited in claim 26 wherein said filter material is adapted to at least one of aerodynamically influence said contaminants in said airstream of entrap contaminants such that said contaminants are transiently or permanently captured within said filter material.

28. The energy-transmissible filter as recited in claim 27 wherein at least one transmissible filter comprises:
a filter body having an exterior surface, said filter body defining said interior area to have a predetermined volume;
said filter material permitting said decontamination energy to be substantially transmissible in order to permeate said interior area and said predetermined volume.

29. The energy-transmissible filter as recited in claim 26 wherein said filter material comprises at least one of silicate, quartz, glass, textiles, cellulosic materials, fluorinated polymers, silicon nitride, glass, silver, copper, aluminum, iron, titanium, titanium dioxide, ferric oxide, tin oxide, further comprising solid, woven, planar, folded, or discrete elements.

30. The energy-transmissible filter as recited in claim 26 wherein said decontamination energy comprises at least one of: non-ionizing radiation, ionizing radiation, ultraviolet, infrared, electrons, electrostatics, plasma, light, laser, LED, lamp, excited gas, and similar phenomena which function to effect biological and non-biological contamination within said airstream.

31. The energy-transmissible filter as recited in claim 28 wherein said decontamination energy is generated by a decontamination energy generator that generates said decontamination energy such that it substantially fills or traverses said predetermined volume.

32. The energy-transmissible filter as recited in claim 26 wherein said decontamination energy is generated by a decontamination energy generator that generates said decontamination energy such that it substantially fills or traverses said predetermined volume.

33. The energy-transmissible filter as recited in claim 26 wherein said decontamination energy is applied by a decontamination energy generator, said decontamination energy generator generates ultraviolet or near ultraviolet spectrum between 10-420 nm wavelength.

34. The energy-transmissible filter as recited in claim 33 wherein at least one of said filter material or said internal area comprises a material that permits said spectrum to be substantially transmissible therethrough.

35. The energy-transmissible filter as recited in claim 26 wherein filter material comprises a hydrophilic surface with a water contact angle of less than about 40 degrees.

36. The energy-transmissible filter as recited in claim 26 wherein filter material comprises a hydrophilic surface having a surface tension greater than 40 dynes/cm in order to facilitate increasing at least one of a surface-bioaerosol interaction or an exposure or dwell time within said electromagnetic field.

37. The energy-transmissible filter as recited in claim 35 wherein filter material comprises a hydrophilic surface having a surface tension greater than 40 dynes/cm in order to facilitate increasing at least one of a surface-bioaerosol interaction or an exposure or dwell time within said electromagnetic field.

38. The energy-transmissible filter as recited in claim 26 wherein said filter material comprises a surface adapted to have a predetermined water contact angle of about 40 degrees or less.

39. The energy-transmissible filter as recited in claim 26 wherein said filter material comprises a surface tension of at last 40 dynes/cm.

40. An energy-transmissible air filtration system comprising:
at least one mechanical filter; and
at least one energy source operatively associated with said at least one mechanical filter for generating an electromagnetic energy wave or force;
wherein said electromagnetic energy wave or force traverses inside at least one material said filter, resulting in simultaneous mechanical filtration and energy-based decontamination of an airstream as said airstream passes through said filter.

41. The energy-transmissible air filtration system according to claim 40 wherein said at least one mechanical filter comprising at least one transmissible filter surface functioning to aerodynamically influence or entrap air contaminants such that said contaminants are transiently or permanently captured within said filter;
said mechanical filter further comprising a filter exterior and a filter body comprising a filter volume, said volume being substantially transmissible to said electromagnetic energy such that said radiation substantially permeates said volume.

42. The energy-transmissible air filtration system according to claim 40 wherein said filter comprising materials including silicate, quartz, glass, textiles, cellulosic materials, fluorinated polymers, silicon nitride, glass, silver, copper, aluminum, iron, titanium, titanium dioxide, ferric oxide, tin oxide, further comprising solid, woven, planar, folded, or discrete elements.

43. The energy-transmissible air filtration system according to claim 40 wherein said electromagnetic energy comprising non-ionizing radiation, ionizing radiation, ultraviolet, infrared, electrons, electrostatics, plasma, light, laser, LED, lamp, excited gas, and similar phenomena which function to effect biological and non-biological contamination within an airstream.

44. The energy-transmissible air filtration system according to claim 40 wherein said electromagnetic energy is detectable throughout substantially all of said filter volume, creating an electromagnetic field substantially filling or traversing said filter volume.

45. The energy-transmissible air filtration system according to claim 40 wherein said electromagnetic radiation comprising the ultraviolet or near ultraviolet spectrum between 10-420 nm wavelength.

46. The energy-transmissible air filtration system according to claim 40 wherein said at least one transmissible filter surface comprising a substantially hydrophilic surface with a water contact angle less than 40 degrees and/or a critical surface tension greater than 40 dynes/cm. Said hydrophilic surface increasing surface-bioaerosol interaction, exposure, and/or dwell time within said electromagnetic field.

47. The energy-transmissible air filtration system according to claim 40 wherein said filter volume comprising a material substantially transmissible to said spectrum.
